(19) United States
(12) Patent Application Publication
(10) Pub. No.: US 2026/0216424 A1
(43) Pub. Date: Jul. 30, 2026

(54) SYSTEM AND METHOD FOR DELIVERY MONITORING AND OCCLUSION DETECTION IN AN INFUSION PUMP

(71) Applicant: Deval KARIA, Kolkata (IN)

(72) Inventor: Deval KARIA, Kolkata (IN)

(21) Appl. No.: 19/141,845

(22) PCT Filed: Dec. 20, 2023
(86) PCT No.: PCT/IB2023/063009

§ 371 Date: Jun. 20, 2025

(30) Foreign Application Priority Data
Dec. 20, 2022 (IN) 202231073969

(51) Int. Cl. A61M 5/145; A61M 5/168

(52) U.S. Cl. CPC A61M 5/14566; A61M 2005/16868; A61M 2205/332

(57) ABSTRACT

System and method for delivery monitoring and occlusion detection in an infusion pump The present disclosure provides systems and methods for delivery monitoring and occlusion detection in an infusion pump and uses thereof. The systems of the present invention are fast and can be modular. The systems and methods allow for direct, accurate, precise, sensitive, and non-invasive measurement of the force required to displace a plunger in an infusion pump.

### FIELD OF THE INVENTION

The present invention relates to a system and method for delivery monitoring and occlusion detection in an infusion pump. The system and method of the present invention are modular and allow for direct, accurate, precise, sensitive and non-invasive measurement of the force required to push the plunger and expel the drug from the reservoir in an infusion pump.

### BACKGROUND OF THE INVENTION

Most commercially available infusion systems work on the syringe pump principle, wherein the controlled movement of a plunger within a syringe dispenses the desired volume of a drug. The plunger is actuated or displaced by a piston or piston rod, linear movement of which is achieved via a lead screw and nut combination. Precise rotation of one (lead screw or nut) allows for a corresponding linear motion of the other component (coupled to the piston), which then actuates the plunger. Pumps often differ in methods of rotational actuation of the lead screw and nut combination. Conventional pumps typically make use of a drive motor assembly (gearbox, motor, encoder) for achieving precise rotation of the screw and nut. Patients' choice of drug volume, in turn, drives the quantum of drive motor assembly set rotation.

The drug reservoir (essentially a disposable syringe) is housed or held within the pump and is connected to an infusion set. The infusion set delivers the drug to the patient via a cannula. The reservoir and infusion set constitute the delivery pathway and are disposable to allow for sterility.

Occlusion can occur gradually because of progressive factors or can occur acutely. Progressive occlusion can be due to either an actual obstruction of the infusion set caused by fibrils (such as insulin fibrils) at the cannula outlet or in the tubing, mechanical causes (e.g., a clamped line or closed stopcock), drug or mineral precipitates, or lipid residue, drug incompatibilities in co-infused drugs (multiple drugs delivered through same line) progressive kinking of the cannula, compression of the skin around the infusion site due to local inflammation or a hematoma at the insertion site, or displacement of the infusion set (https://www.ncbi.nlm.nih.gov/pme/articles/PMC5505439/). In either progressive or acute occlusion, a predictive algorithm can analyse the rise of pressure and deliver an occlusion alarm. Occlusions in an infusion pump can result in an inaccurate amount of drug being administered in a patient, leading to adverse events (AEs).

Occlusion detection by infusion pumps may depend on detecting increased pressure in the tubing, increased radial diameter of the tubing, lack of a temperature gradient of heated fluid, lack of optical signals from an inline piston, or an unexplained rise in the concentration of interstitial fluid glucose or biomarker. Various occlusion detection technologies and systems are known in the art. For instance, U.S. Pat. No. 5,695,473A provides a method and system for monitoring pressure conditions in the fluid pressure upstream and downstream of an intravenous fluid administration system and detecting upstream and/or downstream occlusions. A method for detecting pressure conditions at an upstream or downstream detector across the wall of the tubing to identify upstream or downstream occlusions, respectively is disclosed in this document. This document also provides a process for determining the fluid pressure in a fluid tube across the tube wall where the normal tube force changes over time. Also provided is a process which uses normalization process to eliminate changes in the tubing resilience.

In US patent No.: U.S. Pat. No. 9,889,254B2, an injection device for injecting automatically a medicament is disclosed which comprises an occlusion detection system with a force measurement unit and two separate occlusion detectors. The occlusion detectors take force measurements at different respective measurement rates and generate occlusion indicating signals based on a set of force measurements taken at the respective measurement rates. In US patent No.: U.S. Pat. No. 9,833,561B2, a system, method, and apparatus to detect abnormalities in delivery of a fluid are disclosed which include an infusion apparatus that is controllable to cause one or more perturbations in a fluid flow, a force signal representative of the perturbed force response used to determine an integrated perturbed force response value. A ratio between the integrated perturbed force response value and a normalizing value is used to determine if fluid flow is occluded. In US patent No.: U.S. Pat. No. 6,485,465B2, an occlusion detection system is disclosed, which detects an occlusion in a fluid path of an infusion pump. The infusion pump disclosed in this patent document includes a housing, a motor, a reservoir, one or more drive train components, a sensor and an electronics system. The one or more drive train components react to stimulus from the motor to force fluid from the reservoir into the user. The sensor is positioned to measure a parameter associated with the motor or a drive train component, and the sensor produces three or more output levels across a range of measurements. The electronics system processes the sensor output levels to declare when an occlusion exists. In US patent No.: U.S. Pat. No. 9,610,404B2, an infusion pump system is disclosed, which includes an occlusion detection system to detect when an occlusion exists in the fluid path between the medicine reservoir and the infusion site location. The occlusion detection system can be configured to self-calibrate in a manner that accounts for changes in environmental conditions, such as ambient temperature, pressure, or the like so that the occlusion detection system provides reliable feedback to a user as to the occluded or non-occluded state of the medicine flow path.

In US patent application No.: US20130245595A1, a device for delivering fluid to a user is disclosed which includes a housing, a drive motor assembly in the housing, a force sensor, and an electronics module. The drive motor assembly regulates delivery of fluid by actuating a piston of a fluid reservoir. The electronics module processes the output levels of the force sensor to assess the operating health of the force sensor, to check for occlusions in the fluid delivery path, and to monitor the seating status of the fluid reservoir. The method and system in US patent application US20130245595A1 make use of the force sensor to detect changes in fluid pressure. The force sensor in the system, disclosed in this document, is positioned behind the drive motor assembly. The lead screw is placed at the tip of the drive motor assembly, and the nut thread is present on the inside surface of the piston. With such an arrangement, the force applied to the force sensor by the drive motor assembly is proportional to the pressure applied to the drug due to the power supplied to the drive motor assembly to advance the plunger via the piston. This method and system required the entire drive motor assembly to be displaced to detect changes in the force sensor. It is relatively insensitive to small changes in applied pressure and can only detect significant changes in said pressure, leading to significantly high time for occlusion detection, missed drug delivery and AEs.

In Canadian Patent No.: CA2029267C, method and system for detecting an occlusion in a fluid line upstream of a pump concerns a fluid pump with a pump cycle in which upstream pressure of the fluid line is communicated to downstream portion of the fluid line. A pressure sensor is located downstream of the pump which is capable of measuring a relatively large negative-going pressure in the fluid line. A signal is generated to indicate occlusion based upon detection of negative-going pressure.

US Patent No.: U.S. Pat. No. 7,875,004B2 relates to systems and methods for detecting an occlusion in a fluid delivery device. The fluid delivery device for delivering therapeutic fluid to a patient disclosed in this document comprises a disposable part comprising a fluid delivery tube; a reusable part comprising at least a first portion of an occlusion detection sensor configured to detect occlusion within a fluid passageway including at least said fluid delivery tube; wherein said fluid delivery tube includes a protrusion extending from said fluid delivery tube, said protrusion is configured to change its orientation with respect to said fluid delivery tube when fluid pressure within said fluid delivery tube increases; said occlusion detection sensor is configured to detect the said change in orientation of said protrusion; and said occlusion detection sensor is operative upon coupling said reusable part and said disposable part.

Most methods known in the art for delivery monitoring and occlusion detection in infusion pumps make use of an indirect indicator or an in-line invasive sensor. In an in-line pressure sensor, a disposable dome with a compliant member allows for rapid changes in the deflection of the compliant member in response to pressure changes. This is then detected in a sensor module optically or using sensitive strain measurement. The sensor is reusable and is removably coupled to the dome. In this pressure sensor, the dome is an in-line, invasive part allowing for direct, accurate measurements. However, this part must be bio-compatible for sterile applications and must be disposable or sterilizable to accomplish this. It also warrants the user to replace this part before each use and must also be specifically designed for different tube sizes of corresponding infusion sets.

Also known in the art are methods and systems which use changes in the radial diameter of the tubing. In such methods, a sensitive strain measurement or optical sensor monitors changes in the diameter of relatively compliant tubing. As occlusion occurs, the pressure within the tubing builds up, increasing the diameter of the tubing. This change in diameter can be calibrated to detect normal delivery vs. occlusion. This indirect occlusion detection requires calibration or adjustment for different tubing sizes and materials. Such systems can then only work with such specified/pre-determined tubing and potentially lead to false alarms in case of diameter change due to tube kinking. Additionally, the tubing being compliant makes it inherently prone to kinking.

Another method and system known in the art use change in current drawn by a motor. In such methods, a lead screw and nut combination are directly coupled to a drive motor assembly (gearbox, motor, encoder). Motor rotation leads to linear motion of the piston and plunger, which then expels the drug. The current drawn by the motor is directly proportional to the torque required to rotate the lead screw and/or nut. In the occluded state, this torque is higher than in normal operation, leading to a higher current being drawn by the motor. The change in current drawn can be calibrated to adjust for normal delivery vs. occlusion. This method indirectly indicates occlusion and requires calibration and/or adjustment for different motors, tubing sizes, material, and dispensed volume, among other items. This is a coarse method, which allows for distinction between two states (normal vs. occlusion). Further refinement and/or anomaly detection is often difficult.

None of the methods or systems known in the art is non-invasive while allowing for direct, accurate, precise, and sensitive measurement of the force required for pushing the plunger and expelling the drug from the reservoir. Thus, there is an unmet need in the art for such a system and method, which can then allow for delivery assessment and detection or prediction of occlusion with high speed, accuracy, and repeatability. There is also a need in the art for a modular system to provide the aforementioned advantages in existing infusion pumps by retrofitting them.

### OBJECTS OF THE INVENTION

Accordingly, it is an object of the present invention to provide a system and method which provides direct, accurate, precise, sensitive and non-invasive method of force required to push a plunger. Another object of the present invention is to provide a system and method for delivery assessment and detection/prediction of occlusion in an infusion pump. Yet another object of the present invention is to provide a modular system which can be retrofitted into existing systems as an add-on/accessory. Still, another object of the present invention is to provide a system and method wherein monitoring of provided forces significantly improves the time for occlusion detection.

### SUMMARY OF THE INVENTION

The present invention provides a system and method for direct, sensitive, accurate, precise and non-invasive method of force required to push a plunger/piston which allows for delivery assessment and detection/prediction of occlusion in an infusion pump. The system of the present invention is modular and can be retrofitted into existing systems as an add-on/accessory. The method and system result in monitoring of plunger forces and leads to a significant improvement in time for occlusion detection, reduction in AEs which is a key aspect governing patient safety.

In an embodiment, the present invention provides a system for delivery monitoring and occlusion detection in infusion pump, comprising:
at least one force transmission member (1) comprising at least two ends;
at least one plunger (2) or piston;
at least one force sensor (3);
   wherein at least one end of said force transmission member (1) is facing said plunger (2) or piston and at least one end is facing said force sensor (3);
   wherein said force sensor (3) is placed close to or in immediate contact with the force transmission member.

In a further embodiment, the present invention provides that force sensor can be placed in a manner selected from one or more of horizontally, vertically or concentric circles, concentric spheres, concentric regular polyhedral or concentric regular polygons in relation to said force transmission member (1) in the system.

In a further embodiment, the present invention provides that the force transmission member (1) in the system is capable of displacement from its position.

In another embodiment, the present invention provides that the plunger (6) has at least one end facing a barrel body (6) and at least one end facing the force transmission member (1).

In a still further embodiment, the present invention provides that the sensor (3) is further in contact with at least one force sensor holder (4).

In a yet further embodiment, the present invention provides that the system further comprises at least one piston member (5).

In a yet another embodiment, the present invention provides that the force transmission member (1) in the system is optionally rigidly coupled to the piston member (5).

In a further embodiment, the present invention provides that the piston member (5) optionally comprises at least one compliant member (7).

In an optional embodiment, the present invention provides that the system comprises additional sensors placed close to or in contact with the force transmission member (1).

In another embodiment, the present invention provides that the system further comprises—a computational module configured to analyze force data for indicators of contact detection between the force transmission member and the piston or plunger in an infusion pump during priming, correct or incorrect reservoir seating, confirm the reservoir seating, and to distinguish between normal and occluded delivery in an infusion pump.

In one of the embodiments the present invention provides a modular force sensing unit capable of retrofitting in an infusion pump for allowing delivery monitoring and occlusion detection, comprising:
at least one force transmission member (1) comprising at least two ends;
at least one force sensor (3); and
at least one mounting member (8) configured to allow retrofitting of the modular system in an infusion pump;
   wherein at least one end of said force transmission member (1) is facing plunger (2) or piston of said infusion pump and at least one end is facing said force sensor (3);
   wherein said force sensor (3) is placed close to or in immediate contact with the force transmission member (1).

In another embodiment the present invention provides that in the modular force sensing unit the force sensor can be placed in a manner selected from one or more of horizontally, vertically or as concentric circles, concentric spheres, concentric regular polyhedral or concentric regular polygons in relation to said force transmission member (1).

In another embodiment the present invention provides that the modular force sensing unit further comprising plurality of piston add-ons.

In another embodiment the present invention provides that the modular force sensing unit optionally comprises additional sensors placed close to or in contact with the force transmission member (1).

In another embodiment the present invention provides that the modular force sensing unit further comprising a computational module configured to analyze force data for indicators of contact detection between the force transmission member and the piston or plunger in an infusion pump during priming, correct or incorrect reservoir seating, confirm the reservoir seating, and to distinguish between normal and occluded delivery in an infusion pump.

In one of the embodiments the present invention provides a method for delivery monitoring and occlusion detection in an infusion pump comprising:
i) pushing at least one plunger or piston by at least one force transmission member further in syringe barrel of the infusion pump during each actuation;
ii) transmitting force from the plunger or piston to at least one force sensor by the force transmission member during each actuation;
iii) capturing force required by force transmission member during every additional actuation;
iv) analyzing captured force data and comparing it with pre-set threshold, patterns of force to detect occlusion or any anomaly in normal delivery.

In another embodiment the present invention provides that the method for delivery monitoring and occlusion detection in an infusion pump further comprises additional steps as follows:
v) pausing or stopping drug delivery if occlusion or any delivery anomaly is confirmed; and/or
vi) alerting user by suitable means such as a notification or alarm.

In one of the embodiments the present invention provides a method for contact detection between force transmission member and plunger or piston in an infusion pump during priming comprises one or more of the following steps:
a) initializing system and starting priming sequence followed optionally by calibrating at least one force sensor;
b) beginning movement of at least one force transmission member towards at least one plunger or piston;
c) monitoring data of at least one force sensor;
d) analyzing data of at least one force sensor to detect contact; and
e) determining plunger position based on piston movement.

In another embodiment the present invention provides that the method for contact detection between piston and plunger in an infusion pump during priming, further comprising the following optional step:
f) cross-verifying estimated drug volume with expected drug volume based on reservoir specifications and initiating error handling procedures in case of significant discrepancies.

In one of the embodiments the present invention provides a method for monitoring reservoir seating in an infusion pump comprising one or more of the following steps:
a) Starting priming sequence followed by movement of at least one force transmission member towards at least one plunger or piston to initiate contact;
b) Monitoring data of at least one force sensor;
c) Recoding force value at point of contact of the piston and the plunger; and
d) Analyzing force data for patterns indicative of reservoir seating correctness.

In another embodiment the present invention provides that the method monitoring reservoir seating in an infusion pump, further comprising the following optional step:
e) Making decision on reservoir seating based on the force pattern analysis of step (d) and initiating manual inspection or re-seating of the reservoir in case of indication of potential seating issues.

In one of the embodiments the present invention provides a method for distinguishing between normal and occluded delivery in an infusion pump comprising one or more of the following steps:
i) initializing system for delivery;
ii) actuating at least one piston;
iii) monitoring and analyzing force data; and
iv) Identifying and responding to potential occlusion based on force data deviations.

In one of the embodiments the present invention provides an apparatus for delivery-monitoring and occlusion detection comprising the modular force sensing unit of the present invention.

In one of the embodiments the present invention provides use of system, modular force sensing unit, apparatus and/or methods of the present invention for delivery-monitoring and occlusion detection during fluid flow in an infusion pump.

### DESCRIPTION OF THE DRAWINGS

FIG. 1a shows section view of a portion of the system within the piston. The drug reservoir is in direct contact with the sliding member. FIG. 1b shows section view of the system within the piston. The drug reservoir is in direct contact with the sliding member.

FIG. 2 shows section view of a portion of system within the piston. The drug reservoir is in direct contact with the sliding member. The sliding member is coupled to a compliant mechanism for force amplification or curtailment.

FIG. 3 shows section view of a portion of system within the piston. The drug reservoir is in direct contact with the sliding member. The sliding member is coupled to a compliant mechanism for force direction change.

FIG. 4 shows force vs. time during delivery—with and without occlusion-bolus (1.5 U/min.) measured using the system of invention.

FIG. 5 shows force vs. time during delivery—with occlusion-basal (1 U/hour) measured using the system of invention.

FIG. 6a shows modular force or occlusion sensing unit that can be retrofitted into existing infusion pumps. FIG. 6b shows cross section view of modular force or occlusion sensing unit that can be retrofitted into existing infusion pumps.

FIG. 7 shows how the modular force or occlusion sensing unit can be clamped onto an existing infusion pump.

FIG. 8 shows illustrative assembly of a piston housing the system of invention with cable for data transmission to a device.

FIG. 9a shows testing setup with magnified image of clamped cannula of an infusion set. The infusion set is inserted into an infusion pump (InsuFlo®) for delivery of a drug. FIG. 9b shows an actual image of the infusion pump.

FIG. 10 shows another section view of a portion of the system within the piston. The drug reservoir is in direct contact with the sliding member.

FIG. 11 shows a flowchart that illustrates an embodiment for detecting contact between the piston and the plunger.

FIG. 12 shows a flowchart that illustrates an embodiment for monitoring reservoir seating.

FIG. 13 shows a flowchart that illustrates an embodiment for distinguishing between normal and occluded delivery.

FIG. 14 shows section view of a portion of the system within the piston. The drug reservoir is in direct contact with the sliding member. The sliding (force transmission) member is attached to the piston by a thin member.

### DESCRIPTION OF THE INVENTION

The present invention provides a system and method for direct, accurate, precise, sensitive and non-invasive method of force required to push a plunger which allows for delivery assessment and detection/prediction of occlusion in an infusion pump. The method, modular unit, and system results in monitoring of plunger forces and leads to a significant decrease in time for occlusion detection, which is a key aspect governing patient safety. Faster occlusion times can avoid adverse events for the patients, for instance hyperglycaemic patients in the case of an insulin pump. The modular force sensing unit of the present invention can be retrofitted into existing systems or infusion pumps as an add-on/accessory.

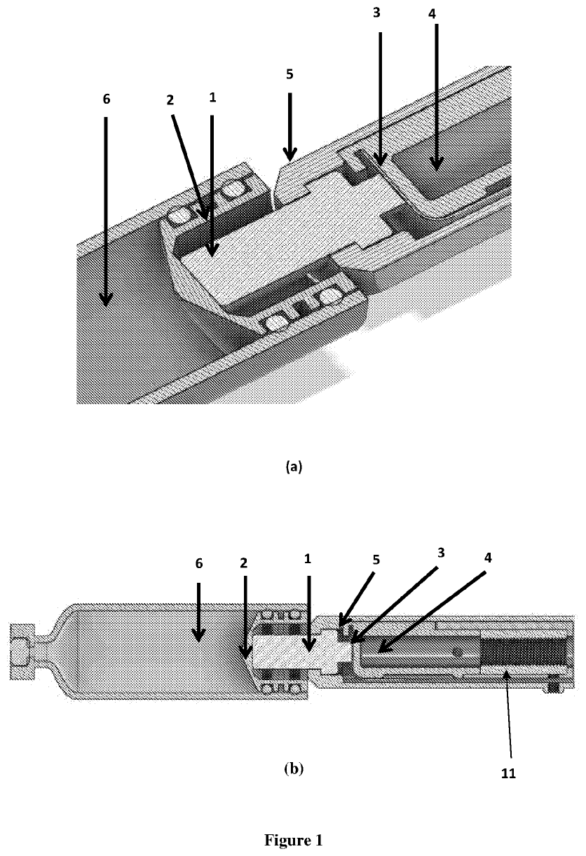

The system or modular unit of the present invention comprises at least one modified piston or piston add-on that incorporates at least one embedded force sensor as shown in FIG. 1. The force sensor can also be incorporated in the system by other means.

The system comprises at least one sliding (force transmission) member (1) which can directly transmit force from at least one plunger (2) to at least one force sensor (3) placed immediately behind said member (1). The placement of force sensor can be immediately behind the force transmission member or sliding member. The placement of force sensor can be in any manner, for instance, concentrically, horizontally, vertically, etc. The force sensor can be placed within piston itself. The placement of the force sensor (3) immediately after the plunger (2) allows for direct measurement of the plunger (2) actuation forces transmitted via the sliding member (1). The sensor (3) is held in place via at least one force sensor holder (4). The force sensor holder (4) is also capable of routing electrical connections from the piston (5) or piston add-on as it travels the stroke length during drug delivery. The system can also comprise one or more nut (11). Alternatively, data transmission from the force sensor (3) can also be undertaken wirelessly. The plunger (2) in most commercially available empty/pre-filled syringes is either a rigid member with a gasket/O-ring placed circumferentially for sealing (as shown in FIG. 1) or a member completely made of a rubber-like/viscoelastic material. The size or dimensions of the sliding member (1) can be scaled/curtailed to match the plunger opening or plunger (2) size. It can be made of materials that are sufficiently rigid to allow accurate force transmission. The force sensor (3) can be a standard one available in the art or can be customized for a particular application; when parameters of a standard sensor are insufficient to meet the need (ex. range of force measurement, response time etc.). Standard force sensors are essentially force-sensitive resistors, based on the principal of change in resistance being proportional to the force applied to it. With each actuation the sliding member (1) pushes the plunger (2) further in barrel body (6) and captures the force required during each actuation via the force sensor (3). Analysis of the captured force data and its comparison with pre-set thresholds/patterns of force can help discern between normal delivery and occlusion. High-resolution analysis of the captured force data can also be used for anomaly detection and prediction of occlusion events. This can warn the patient or user prior to complete occlusion, allowing for corrective measures to be undertaken, significantly reducing occurrence chances of AEs.

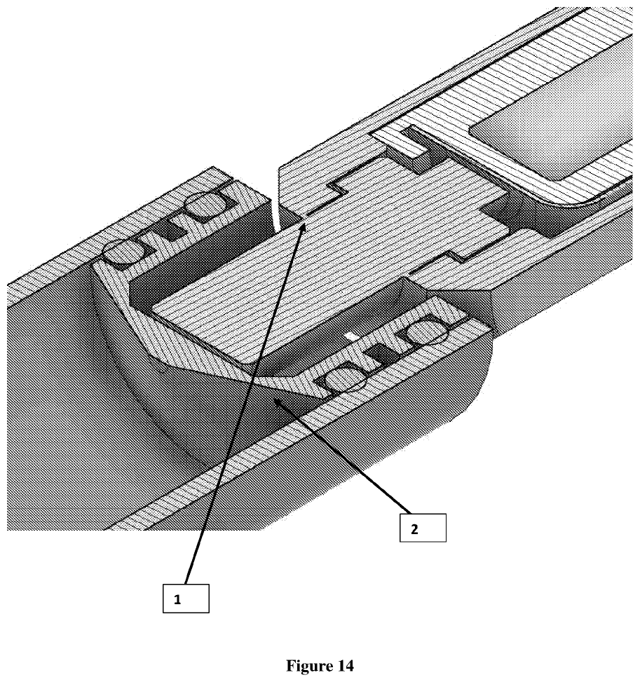

In another embodiment, the sliding member (1) is modified to be rigidly coupled to the piston (5) (FIG. 14) via a thin connection around the circumference of the sliding member (1), allowing for the sliding member (1) to move linearly for force transmission, while protecting it from ingress. Only the sliding embodiment (without the thin connection) can be used when ingress protection is not critical.

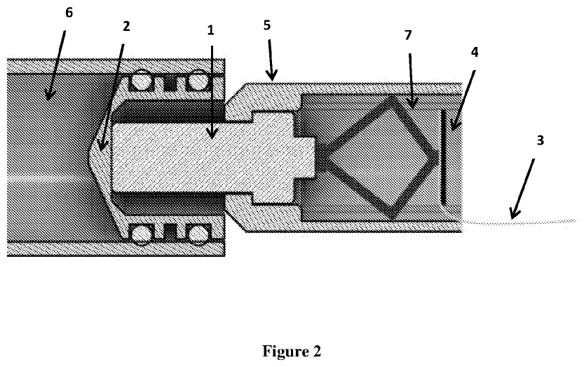

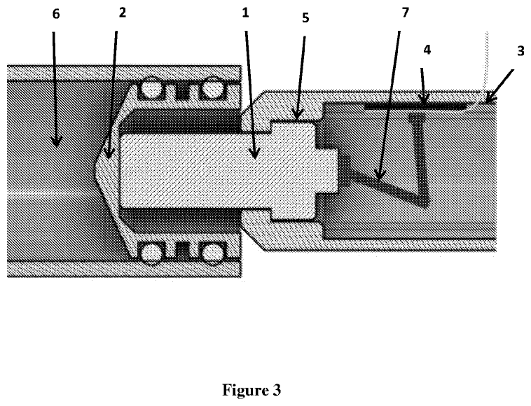

At least one compliant mechanism (7) within the piston member, coupled to the sliding member can also allow for force/displacement change to suit the force sensor range/size constraints (FIG. 2, FIG. 3). This allows a wide range of sensors to be used and sensitivity of measurement to be tuned. Additionally, a lead screw and/or nut (FIG. 1b) can be incorporated for linear motion to allow for a compact design.

Additional sensors, non-limiting examples including, sensors for temperature and humidity can also be incorporated close to/in contact with the sliding member (1) which can then be used to measure additional parameters of the drug to be administered for example, estimating temperature of the drug which is critical for thermally sensitive drugs. In case of using one or more temperature sensors for the temperature measurement, the sliding member (1) and/or plunger (2) should be made of thermally conductive material such as metals and a temperature sensor must be placed in addition to a force sensor. The placement of additional sensor can be one or more of besides, front, behind, up or down of the force sensor. When the sliding member comes in contact with the plunger (2) (which is in contact with the drug or fluid of which temperature is to be measured) then the temperature sensor can capture the data. Sensitivity of measurement of the temperature can be changed by changing the nature of material used for making the sliding member (1) and/or plunger (2) thereby changing their thermal conductivity.

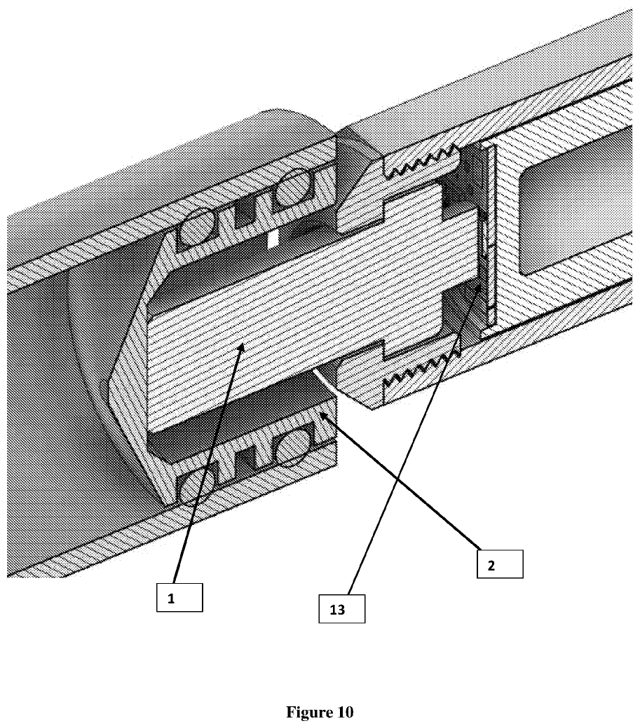

In another embodiment, integrated sensors (temperature, pressure, humidity) (FIG. 10) can also be used to provide multiple streams of data which can be calibrated based on the desired application.

The entire system/method can be made into a modular, standalone device which can be retrofitted into existing systems as an add-on/accessory. Due to the novel placement of the force measurement sensor, the results from such a system in terms of sensitivity, anomaly detection etc. are expected to be significantly better than the standard methods of delivery monitoring/occlusion detection employed in the art (such as current spike detection etc.). At least one mounting member (8) can be used in the modular unit which can be designed in different configurations (such Snap-on, screw-on etc.) depending on the mounting space availability in the existing system. The data from the force sensor can be collected wirelessly or wired into a DAQ system.

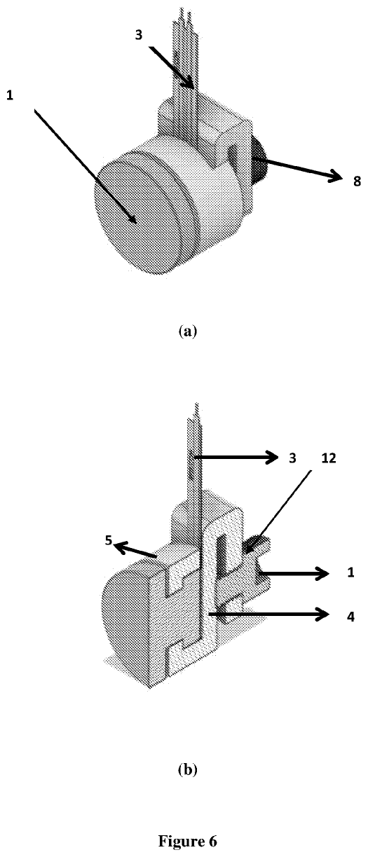

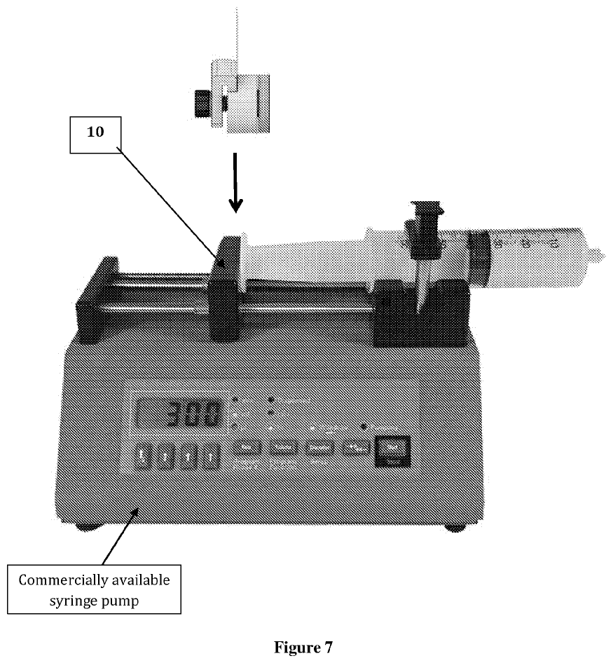

The modular force sensing unit of the present invention is capable of retrofitting in an infusion pump for allowing delivery monitoring and occlusion detection (FIGS. 6 and 7). Such infusion pumps permit mounting of a commercially available stock syringe (comprising at leaste one of needle coupling port, barrel, piston and plunger) using a fixed syringe holder block and a movable pusher block (10), position of which can be adjusted based on the total syringe length after filling of the drug. The end of the piston is then displaced/actuated by the pusher block (10), which may be available with at least one anti-siphon plate (not shown in Figures) to expel the drug. The modular force sensing unit can be mounted onto the pusher block, anti-siphon plate and comprises at least one sliding (force transmission) member (1) comprising at least two ends and at least one force sensor (3). The sliding member (1) has at least two ends wherein at least one end is facing piston of the mounted syringe and at least one end is facing said force sensor (3). The force sensor (3) is in contact and placed concentrically in relation to said sliding member (1). The placement of the force sensor (3) can be immediately behind the force transmission member or piston of the mounted syringe. The placement of force sensor can be in any manner, for instance, concentrically, horizontally, vertically, etc. The force sensor can be placed within piston itself. The specific placement of force sensor allows for direct measurement of the piston actuation forces transmitted via the sliding member (1). The modular unit comprises at least one mounting member (8) configured to allow retrofitting of the modular system in an infusion pump, typically mounted onto the pusher block, anti-siphon plate. The modular unit can also comprise one or more of screw (12) for allowing its mounting in existing pumps. The sliding member (1) size can be scaled or curtailed to match the piston size of the mounted syringe. It must also be made of rigid materials to allow accurate force transmission. The force sensor (3) can be a standard one available in the art or a custom sensor designed for a particular application; when parameters of a standard sensor are insufficient to meet the need (ex. range of force measurement, response time etc.). Standard force sensors are essentially force-sensitive resistors, based on the principal of change in resistance being proportional to the force applied to it.

The placement of the force sensor is an important aspect of the present invention. The placement of the force sensor immediately after the plunger or piston allows for direct measurement of the plunger or piston actuation forces transmitted via the sliding member. Such direct, non-invasive measurement provides sensitive, accurate, and precise data of force required to push a plunger or piston leading to a significant improvement in time until occlusion detection, and accuracy of occlusion detection, which are key aspects governing patient safety. Known prior art systems which use a force sensor, place it much further away from the plunger or piston (typically after the drive motor assembly) which makes them insensitive, and leads to mechanical design complications (due to the number of intermediate parts), and additional calibration/algorithms to determine force values while not providing faster or accurate results.

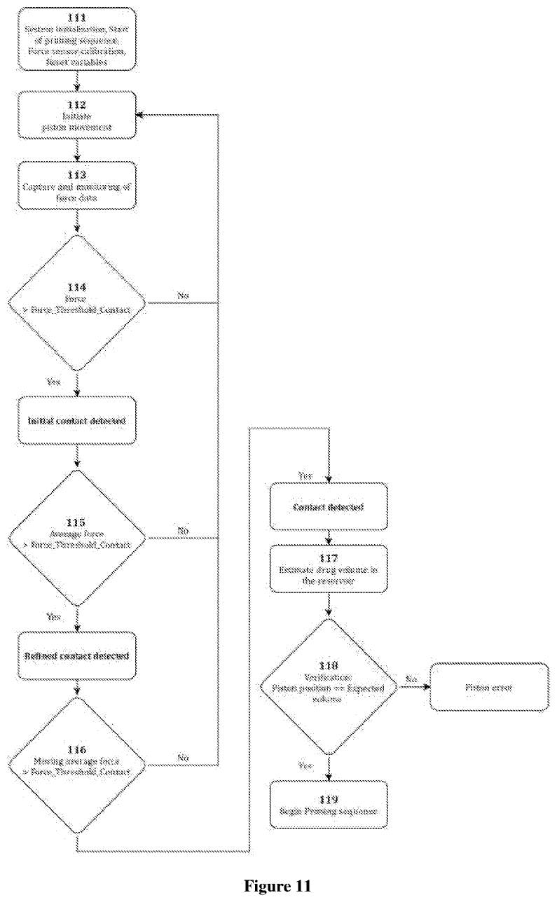

FIG. 11 illustrates a flow chart for an embodiment of a process utilized in a fluid infusion device such as InsuFlo™, for detecting contact between the piston and the plunger during the priming sequence. This process is critical for ensuring accurate preparation for drug delivery.

The process begins with system initialization, starting the priming sequence and calibrating the force sensor (task 111). Subsequently, the piston starts moving towards the plunger at a controlled speed (task 112).

Force sensor data is continuously monitored by the device (task 113). Initial contact is detected by a significant increase in force readings, surpassing the Force_Threshold_Contact (task 114). This threshold is empirically determined and adjusted based on the piston, plunger material characteristics, environmental conditions, drug type and expected backpressure among other factors.

The process refines contact detection by employing a buffer to store recent force readings and calculating their average (task 115). Stable contact is confirmed if the average force consistently exceeds Force_Threshold_Contact for a specified duration (task 116).

Plunger position is then determined by calculating the distance moved by the piston from priming start to stable contact, estimating the drug volume in the reservoir (task 117).

A verification step cross-checks the estimated drug volume against expected values, flagging discrepancies and prompting for manual verification if needed (task 118).

Upon successful verification, the pump continues the priming sequence (task 119). It may be obvious to a person skilled in the art that thresholds, patterns, and techniques utilized in data analysis could be adapted to different environmental conditions, drug type, and expected backpressure among other factors. Sample data analysis techniques would include but are not limited to rolling averages, machine learning, median filters among other techniques. These can be utilized for calibration, smoothing out force data, reducing noise and false positives.

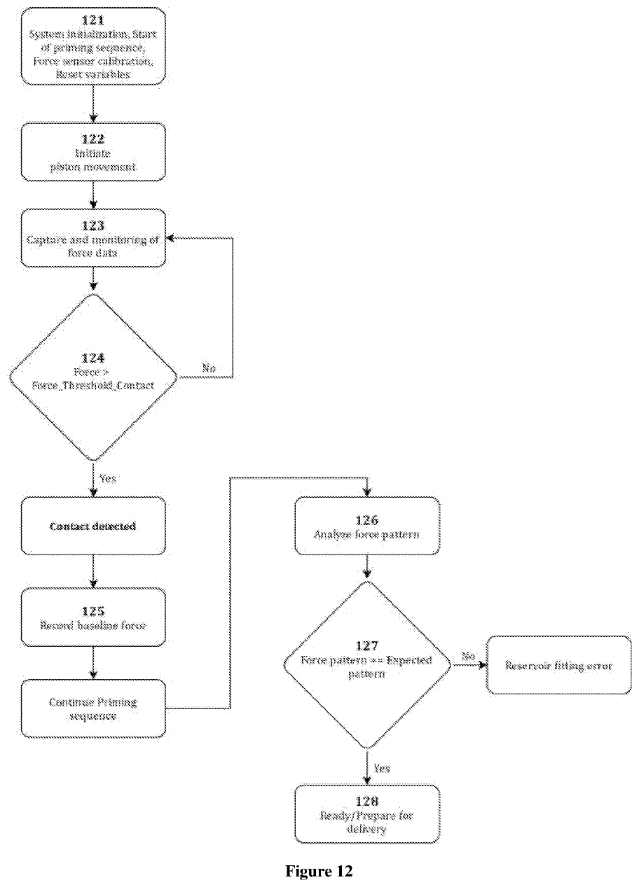

FIG. 12 is a flow chart illustrating an embodiment of a process that monitors reservoir seating in a fluid infusion device, such as the InsuFlo™ device described herein. The process utilizes force sensor data to determine the correctness of the reservoir seating during the priming sequence. Initially, the process commences with the start of the priming sequence (task 121), wherein the piston is moved towards the plunger to initiate contact (task 122).

As the piston progresses towards the plunger, the process continuously monitors force sensor data (task 123). This data is critical for detecting the initial contact between the piston and the plunger, characterized by a noticeable increase in force (task 124). Upon this detection, the force value(s) at the point of contact is recorded as the baseline force (task 125).

The process proceeds with the priming sequence until fluid is visibly expelled from the infusion set. During this phase, the force data is analyzed for patterns indicative of reservoir seating correctness (task 126). Correct seating typically shows a gradual, consistent increase in force, while incorrect seating may exhibit erratic patterns or unusual force spikes.

The decision on reservoir seating is made based on the force pattern analysis (task 127). If the observed pattern aligns with the expected pattern for correct seating, the reservoir is deemed correctly seated. Otherwise, potential seating issues are indicated, prompting manual inspection or re-seating of the reservoir.

Upon confirming correct reservoir seating, the process completes the priming process and readies the device for normal delivery (task 128). The algorithm concludes following the verification of correct reservoir seating or upon the indication of a need for manual intervention.

It may be obvious to a person skilled in the art that to enhance accuracy, the process may incorporate statistical analysis and machine learning for advanced pattern recognition in force data. Additionally, force thresholds may be set based on empirical data to help distinguish between normal and abnormal force readings during priming, aiding in the early detection of seating anomalies and potential occlusions before delivery begins to the user. Thresholds, patterns, and techniques utilized in data analysis could be empirically determined, adapted to different environmental conditions, drug type, and expected backpressure among other factors.

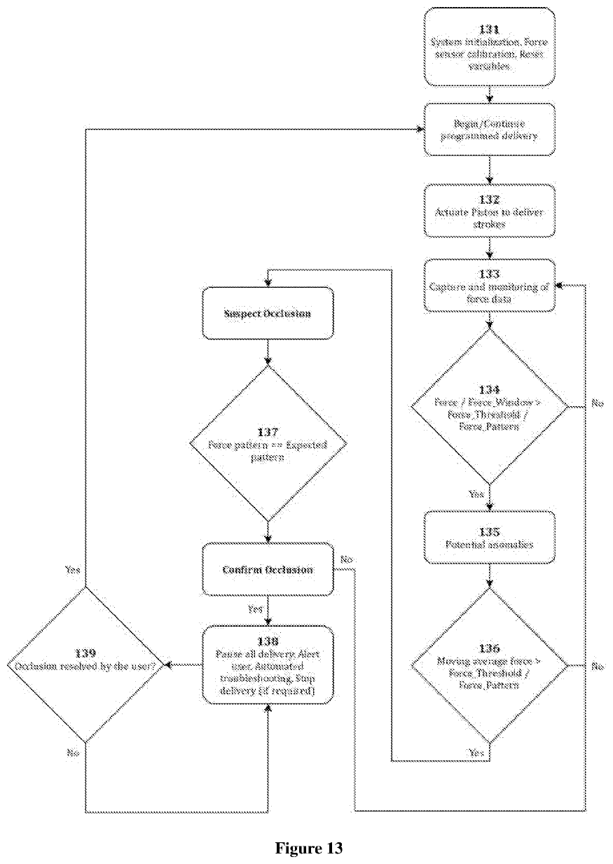

FIG. 13 presents a flow chart illustrating an embodiment of a process for distinguishing between normal and occluded drug delivery in a fluid infusion device, like InsuFlo™. This process leverages force sensor data to differentiate between these delivery states in either Basal or Bolus modes.

The process begins by initializing the system for delivery, resetting relevant counters, and calibrating the force sensor for accurate measurements (task 131). Following this, drug delivery is initiated as per the programmed mode, with the piston actuating to deliver the drug (task 132).

During delivery, the process continuously monitors real-time force data (task 133). A baseline force pattern for normal delivery is established using historical data (task 134), against which current force data is compared.

Any significant and sustained deviations from the normal force range are flagged as potential anomalies (task 135). If these exceed the predefined occlusion threshold consistently, an occlusion is suspected (task 136).

The process then verifies the potential occlusion, utilizing statistical analysis and machine learning for accuracy (task 137). If occlusion is confirmed, the delivery is paused or stopped, the user is alerted and troubleshooting steps initiated (task 138).

Once the occlusion is resolved, normal delivery resumes with continuous monitoring for further anomalies (task 139). The algorithm concludes following the delivery session or if unresolved occlusion issues require user intervention.

It may be obvious to a person skilled in the art that thresholds, patterns, and techniques utilized in data analysis could be empirically determined, adapted to different environmental conditions, drug type, and expected backpressure among other factors. Sample data analysis techniques would include but would not be limited to rolling averages, machine learning, median filters among other techniques. These can be utilized for calibration, smoothing out force data, reducing noise and false positives.

The various embodiments of present invention will now herein below be described by means of examples. The following examples are provided solely to illustrate the present invention and are not intended to limit the scope of the invention, as described herein.

Examples for representative purpose without limiting scope of the disclosure are illustrated below.

### Example 1: System with Piston and Drug Reservoir in Direct Contact with the Sliding (Force Transmission) Member

The system of the present invention comprises a modified piston or piston add-on that incorporates an embedded force sensor as shown in FIG. 1. The modular system unit of the present invention comprises piston add-ons (not shown in Figures) with an embedded force sensor (FIG. 6). A sliding (force transmission) member (1) directly transmits force from the plunger (2) to the force sensor (3) placed concentrically, immediately behind said member (1). The sensor (3) is held in place via the force sensor holder (4) which also routes the electrical connections from the piston (5) or piston add-on (not shown in Figure) as it travels the stroke length during drug delivery. Alternatively, data transmission from the force sensor (3) can also be undertaken wirelessly. With each actuation the sliding member (1) pushes the plunger further and captures the force required during this actuation via the force sensor (3). Analysis of the captured force data and comparison with pre-set thresholds/patterns of force can help discern between normal delivery and occlusion.

The system of present invention can comprise primary printed circuit board (PCB) comprising one or more of additional sensors (other than force sensor) (FIG. 10). The system within the piston can have drug reservoir in direct contact with the sliding member (FIG. 10). In another embodiment, the system can have the sliding (force transmission) member attached to the piston by a thin member (FIG. 14).

### Example 2: System with the Piston, with the Sliding (Force Transmission) Member Coupled to a Compliant Mechanism

The drug reservoir is in direct contact with the sliding member. The system can comprise piston (5) with sliding (force transmission) member coupled to a compliant mechanism for force amplification or curtailment (FIG. 2). Alternatively, the system can comprise piston with sliding (force transmission) member coupled to a compliant mechanism for force direction change (FIG. 3). The compliant mechanism (7) within the piston member allows for force or displacement change to suit the force sensor range or size constraints (FIG. 2, FIG. 3). This allows a wide range of sensors to be used, and sensitivity of measurement to be tuned.

### Example 3: System with Sensors in Addition to Force Sensor

The system of the present invention can have additional sensors (FIG. 10), non-limiting examples including, sensors for temperature and humidity can also be incorporated close to/in contact with the sliding member (1). In case of using one or more temperature sensors for the temperature measurement, the sliding member (1) and/or plunger (2) should be made of thermally conductive material such as metals and a temperature sensor must be placed in addition to a force sensor. When the sliding member comes in contact with the plunger (2) (which is in contact with the drug of which temperature is to be measured) then the temperature sensor can capture the data. Sensitivity of measurement of the temperature can be changed by changing the nature of material used for making the sliding member (1) and/or plunger (2) thereby changing its thermal conductivity.

In another embodiment, integrated sensors (temperature, pressure, humidity) can also be used to provide multiple streams of data which can be calibrated based on the desired application. Please let me know if you need an image to support these applications.

### Example 4: Modular Force Sensing Unit Capable of Retrofitting in an Infusion Pump

The modular force sensing unit of the present invention is capable of retrofitting in an infusion pump for allowing delivery monitoring and occlusion detection (FIGS. 6 and 7). Such infusion pumps permit mounting of a commercially available stock syringe (comprising of a needle coupling port, barrel, piston, and plunger) using a fixed syringe holder block and a movable pusher block (10), position of which can be adjusted based on the total syringe length after filling of the drug (FIG. 7). The end of the piston is then displaced/actuated by the pusher block to expel the drug. The modular force sensing unit can be mounted onto the pusher block/anti-siphon plate and comprises at least one sliding (force transmission) member (1) comprising at least two ends and at least one force sensor (3) (FIG. 7). The sliding member (1) has at least two ends wherein at least one end is facing piston of the mounted syringe and at least one end is facing said force sensor (3). The force sensor (3) is in contact and placed concentrically in relation to said sliding member (1). The placement of the force sensor (3) immediately after the piston of the mounted syringe allows for direct measurement of the piston actuation forces transmitted via the sliding member (1). The modular unit comprises at least one mounting member (8) configured to allow retrofitting of the modular system in an infusion pump, typically mounted onto the pusher block or the anti-siphon plate. The sliding member (1) size can be scaled/curtailed to match the piston size of the mounted syringe. It must also be made of rigid materials to allow accurate force transmission. The force sensor (3) can be a standard one available in the art or a custom sensor designed for a particular application; when parameters of a standard sensor are insufficient to meet the need (ex. range of force measurement, response time etc.). Standard force sensors are essentially force-sensitive resistors, based on the principal of change in resistance being proportional to the force applied to it.

### Example 5: System with Data Transmission from the Force Sensor Through Wireless Mode

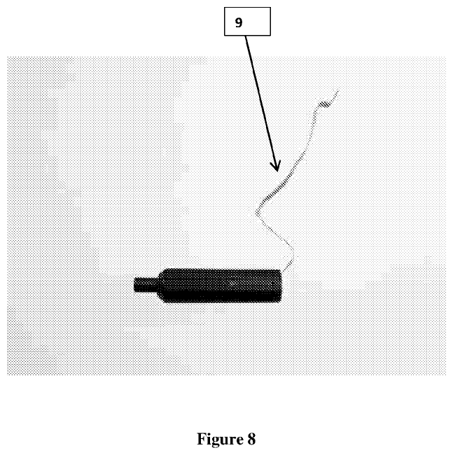

The system can carry out data transmission form the force sensor via wireless mode. This can be implemented via a device (of which the current system is a part thereof). Data from the force sensor is collected in the device's micro-controller and then transmitted along with other data wirelessly. The connection between the device's microcontroller and the force sensor can be done via a cable/wired connected to the primary printed circuit board (PCB) of the device as shown in FIG. 8. This wireless transmission can be via standard methods like Bluetooth, Radio frequency (RF) etc.

### Example 6: Systems, Modular Units and Methods Require Shorter Times for Occlusion Detection

The inventor/applicant of the present invention undertakes experiments to determine time until alarm is triggered after occlusion detection for an affordable insulin pump (InsuFlo™) with the occlusion detection system and method of the present invention and compares it with reported values of a commercially available pump (Medtronic Minimed 722G, https://www.medtronicdiabetes.com/sites/default/files/library/download-library/user-guides/x22_user guide.pdf) that places the force sensor at the end of the drive motor assembly.

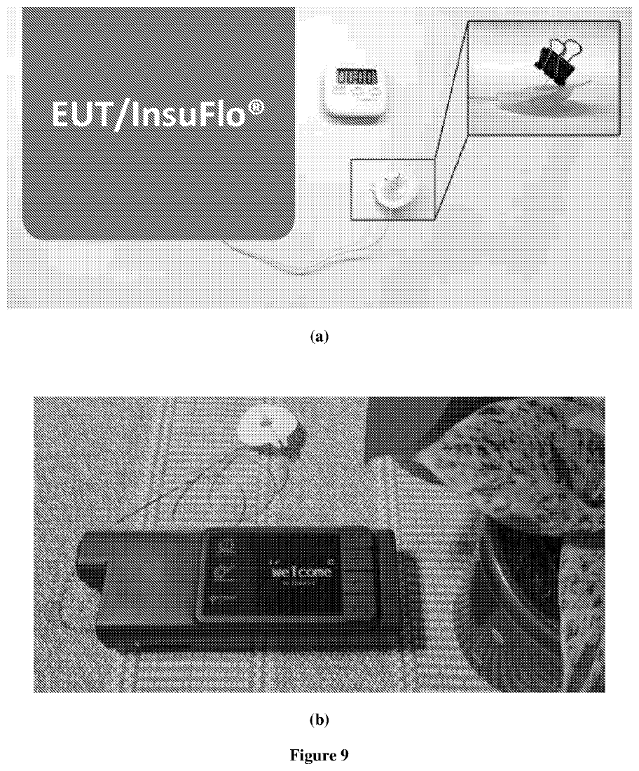

A reservoir connected with an infusion set, containing distilled water was mounted into the equipment under testing (EUT) or InsuFlo™ (FIG. 9). A drug reservoir was seated and an infusion set coupled to the EUT. The infusion set consists at least one tubing ending in an infusion site patch which holds a Teflon cannula, meant for delivery of the drug into the user's tissue. Priming was undertaken as per requisite instructions of the manufacturer. Teflon cannula of the infusion set was then clamped at the tip to simulate an acute or complete occlusion. The EUT was subsequently programmed to deliver a bolus dose (1.5 U/min.), or a basal dose (1 U/hour). Time until an alarm is triggered was noted for five runs. The force threshold for occlusion detection is set at 2.5 N, with the average force during delivery without occlusion observed to be 1.1 N.

Average time until an occlusion alarm is triggered for the EUT was found to be significantly lower than a commercially available device (Medtronic 722 G), for both bolus and basal modes of delivery: 22.4s vs. 116s (1.5 U/min. bolus, average time before alarm), 0.46 hours vs. 3.09 hours (1 U/hour basal, average time before alarm). This was also reflected in the difference between maximum and minimum values of time before alarm (a measure of precision): 8s vs. 70s (1.5 U/min. bolus), 0.1 hours vs. 2.27 hours (1 U/hour basal, average time before alarm) (Table 1, Table 2 below).

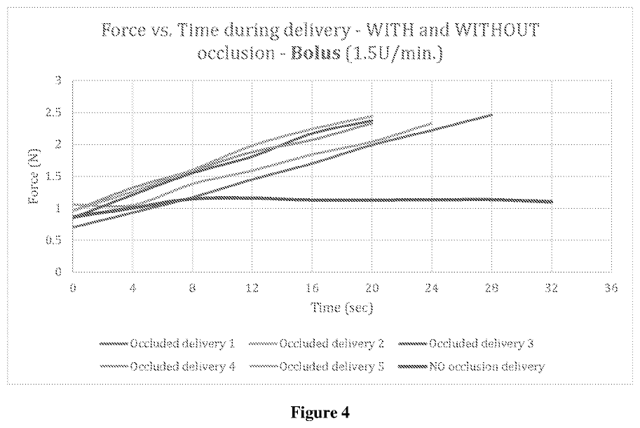

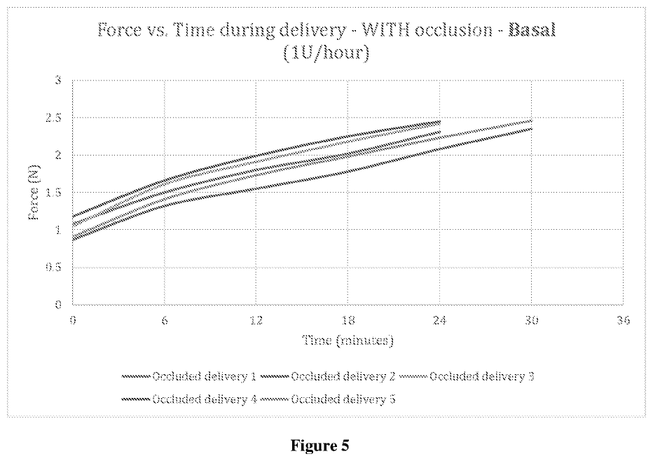

Reported data translates to 80.7% (1.5 U/min. bolus, average time before alarm), 85.1% (1 U/hour basal, average time before alarm) reduction in time for occlusion detection by the EUT (FIG. 4 and FIG. 5). FIG. 4 and FIG. 5 show plots of the force recorded from the system and method of the current invention. This has been shown for each of the five runs of occluded delivery for both bolus and basal modes of delivery. A comparison plot has been provided for normal delivery to highlight the difference between force characteristics. Difference between maximum and minimum values of time before alarm (a measure of precision) also shows an improvement of 88.6% (1.5 U/min. bolus) and 95.6% (1 U/hour basal, average time before alarm).

Significantly shorter times for occlusion detection in the EUT allows for quicker patient intervention and diminished risk of AEs. This can be further improved by incorporating delivery rates and other parameters within the detection algorithm.

TABLE 1
Time for occlusion detection at various delivery
rates for EUT/InsuFlo ™

| Rate | Minimum time | Average time | Maximum time |
|---|---|---|---|
| (EUT/ | before alarm | before alarm | before alarm |
| InsuFlo ™) | (missed insulin) | (missed insulin) | (missed insulin) |
| Bolus delivery | 20 sec | 22.4 sec | 28 sec |
| (1.5 IU/min.) | (0.5U) | (0.56U) | (0.7U) |
| Basal delivery | 0.4 hour | 0.46 hour | 0.5 hour |
| (1 IU/hour) | (0.4U) | (0.46U) | (0.5U) |

TABLE 2
Time for occlusion detection at various
delivery rates for Medtronic Minimed 722G

| Rate | Minimum time | Typical time | Maximum time |
|---|---|---|---|
| (Medtronic | before alarm | before alarm | before alarm |
| Minimed 722G) | (missed insulin) | (missed insulin) | (missed insulin) |
| Bolus delivery | 92 sec | 116 sec | 162 sec |
| (1.5 IU/min.) | (2.3U) | (4.05U) | (2.9U) |
| Basal delivery | 2.2 hours | 3.09 hours | 4.47 hours |
| (1 IU/hour) | (2.2U) | (3.09U) | (4.47U) |

The pump system of the present invention can be utilized in new pump designs to replace conventional piston designs. The system of the present invention also has applications which include a standalone piston add-on/accessory that can be incorporated into existing infusion pumps, laboratory syringe pumps to provide force data for occlusion detection and laboratory research such as micro-fluidic device development.

Although the subject matter has been described herein with reference to certain embodiments thereof, other embodiments within the scope of the present invention are possible. For instance, for illustrative purpose, the systems, modular units and methods of the disclosure comprise force sensor and temperature sensor. However, those skilled in the art would appreciate that scope of the disclosure would extend to the systems, modular units and methods comprising other sensors known in the field of art. It will be obvious to those skilled in the art to make various changes, modifications and alterations to the invention described herein. To the extent that these various changes, modifications, and alteration do not depart from scope of the present invention, they are intended to be encompassed therein.

I claim:

1. A system for delivery monitoring and occlusion detection in infusion pump, comprising:
at least one force transmission member (1) comprising at least two ends;
at least one plunger (2) or piston;
at least one force sensor (3);
wherein at least one end of said force transmission member (1) is facing said plunger (2) or piston and at least one end is facing said force sensor (3);
wherein said force sensor (3) is placed close to or in immediate contact with the force transmission member.

2. The system as claimed in claim 1, wherein the force sensor can be placed in a manner selected from one or more of horizontally, vertically or concentric circles, concentric spheres, concentric regular polyhedral or concentric regular polygons in relation to said force transmission member (1).

3. The system as claimed in claim 1 or 2, wherein said force transmission member (1) is capable of displacement from its position.

4. The system as claimed in claim 1, 2 or 3, wherein said plunger (6) has at least one end facing a barrel body (6) and at least one end facing the force transmission member (1).

5. The system as claimed in any one of claims 1 to 4, wherein said sensor (3) is further in contact with at least one force sensor holder (4).

6. The system as claimed in any one of claims 1 to 5, further comprising at least one piston member (5).

7. The system as claimed in any one of claims 1 to 6, wherein the force transmission member (1) is optionally rigidly coupled to the piston member (5).

8. The system as claimed in any one of claims 1 to 7, wherein the piston member (5) optionally comprises at least one compliant member (7).

9. The system as claimed in any one of claims 1 to 8, wherein the system optionally comprises additional sensors placed close to or in contact with the force transmission member (1).

10. The system as claimed in any one of claims 1 to 9, further comprising:
a computational module configured to analyze force data for indicators of contact detection between the force transmission member and the piston or plunger in an infusion pump during priming, correct or incorrect reservoir seating, confirm the reservoir seating, and to distinguish between normal and occluded delivery in an infusion pump.

11. A modular force sensing unit capable of retrofitting in an infusion pump for allowing delivery monitoring and occlusion detection, comprising:
at least one force transmission member (1) comprising at least two ends;
at least one force sensor (3); and
at least one mounting member (8) configured to allow retrofitting of the modular system in an infusion pump;
wherein at least one end of said force transmission member (1) is facing plunger (2) or piston of said infusion pump and at least one end is facing said force sensor (3);
wherein said force sensor (3) is placed close to or in immediate contact with the force transmission member (1).

12. The modular force sensing unit as claimed in claim 11, wherein the force sensor can be placed in a manner selected from one or more of horizontally, vertically or as concentric circles, concentric spheres, concentric regular polyhedral or concentric regular polygons in relation to said force transmission member (1).

13. The modular force sensing unit as claimed in claim 11 or 12, further comprising plurality of piston add-ons.

14. The modular force sensing unit as claimed in any one of claims 11 to 13, optionally comprising additional sensors placed close to or in contact with the force transmission member (1).

15. The modular force sensing unit as claimed in any one of claims 11 to 14, further comprising:
a computational module configured to analyze force data for indicators of contact detection between the force transmission member and the piston or plunger in an infusion pump during priming, correct or incorrect reservoir seating, confirm the reservoir seating, and to distinguish between normal and occluded delivery in an infusion pump.

16. A method for delivery monitoring and occlusion detection in an infusion pump comprising:
i) pushing at least one plunger or piston by at least one force transmission member further in syringe barrel of the infusion pump during each actuation;
ii) transmitting force from the plunger or piston to at least one force sensor by the force transmission member during each actuation;
iii) capturing force required by force transmission member during every additional actuation;
iv) analyzing captured force data and comparing it with pre-set threshold, patterns of force to detect occlusion or any anomaly in normal delivery.

17. The method as claimed in claim 16, wherein the method further comprises additional steps as follows:
v) pausing or stopping drug delivery if occlusion or any delivery anomaly is confirmed; and/or
vi) alerting user by suitable means such as a notification or alarm.

18. A method for contact detection between force transmission member and plunger or piston in an infusion pump during priming comprises one or more of the following steps:
a) initializing system and starting priming sequence followed optionally by calibrating at least one force sensor;
b) beginning movement of at least one force transmission member towards at least one plunger or piston;
c) monitoring data of at least one force sensor;
d) analyzing data of at least one force sensor to detect contact; and
e) determining plunger position based on piston movement.

19. The method as claimed in claim 18, further comprising the following optional step:
f) cross-verifying estimated drug volume with expected drug volume based on reservoir specifications and initiating error handling procedures in case of significant discrepancies.

20. A method for monitoring reservoir seating in an infusion pump comprising one or more of the following steps:
a) Starting priming sequence followed by movement of at least one force transmission member towards at least one plunger or piston to initiate contact;
b) Monitoring data of at least one force sensor;
c) Recoding force value at point of contact of the piston and the plunger; and
d) Analyzing force data for patterns indicative of reservoir seating correctness.

21. The method as claimed in claim 20, further comprising the following optional step:
e) Making decision on reservoir seating based on the force pattern analysis of step (d) and initiating manual inspection or re-seating of the reservoir in case of indication of potential seating issues.

22. A method for distinguishing between normal and occluded delivery in an infusion pump comprising one or more of the following steps:
i) initializing system for delivery;
ii) actuating at least one piston;
iii) monitoring and analyzing force data; and
iv) Identifying and responding to potential occlusion based on force data deviations.

23. An apparatus for delivery-monitoring and occlusion detection comprising the modular force sensing unit as claimed in any one of claims 11 to 15.

24. Use of system, modular force sensing unit, apparatus and/or methods as claimed in any one of claims 1 to 22 for delivery-monitoring and occlusion detection during fluid flow in an infusion pump.
